(19) United States
(12) Patent Application Publication
(10) Pub. No.: US 2024/0268796 A1
(43) Pub. Date: Aug. 15, 2024

(54) SYSTEM AND METHOD FOR DETERMINING LEVELS OF S-NITROSOTHIOL IN SITU

(71) Applicants: The Trustees of Indiana University, Bloomington, IN (US); Purdue Research Foundation, West Lafayette, IN (US)

(72) Inventors: Benjamin Gaston, Indianapolis, IN (US); Nadzeya Marozkina, Indianapolis, IN (US); Noah Neidigh, West Lafayette, IN (US); Alyssa Alexander, West Lafayette, IN (US); Logan Plack, West Lafayette, IN (US); Parker Van Emmerik, West Lafayette, IN (US); Daniel Cater, Zionsville, IN (US)

(21) Appl. No.: 18/440,359

(22) Filed: Feb. 13, 2024

Related U.S. Application Data
(60) Provisional application No. 63/445,130, filed on Feb. 13, 2023.

(51) Int. Cl. A61B 10/00; A61B 5/00

(52) U.S. Cl. CPC A61B 10/00; A61B 5/0071; A61B 5/7246; A61B 5/7282; A61B 2010/0083

(57) ABSTRACT

Abnormal S-nitrosothiol metabolism is involved in many different diseases, ranging from asthma to septic shock. A critical limitation of current methods for quantifying S-nitrosothiols present in vivo is that samples need to removed be from the body for analysis. The technology of the present disclosure determining S-nitrosothiol levels in situ using ultraviolet light to cleave the S—N bond, releasing the nitric oxide, collecting the nitric oxide, and measuring the nitric oxide.

### CROSS-REFERENCE TO RELATED APPLICATIONS

This application claims priority to U.S. Provisional Patent Application No. 63/445,130 filed on Feb. 13, 2023, which is incorporated by reference in its entirety.

### STATEMENT REGARDING FEDERALLY SPONSORED RESEARCH

This invention was made with government support under grant number HL158507 awarded by the National Institutes of Health—National Heart, Lung, and Blood Institute. The government has certain rights in the invention.

### FIELD

The present disclosure relates to systems and methods for determining levels of metabolites in situ.

### BACKGROUND

S-nitrosothiols are organic compounds containing a nitroso group attached to the sulfur atom of a thiol. S-nitrosothiols constitute small molecules, peptides, and proteins, and they play a role in many different physiological functions, including regulation of respiration, blood pressure, and maintenance of airway smooth muscle tone. Low-mass S-nitrosothiols, such as S-nitroso-L-cysteine and S-nitrosoglutathione, are produced in vivo by metalloproteins and in low-pH environments, and are important signaling molecules. For example, the hemoglobin R to T conformational switch caused by acidosis, hypercapnia, hypoxia, and other physiological phenomena produces bioactive S-nitrosothiols.

Abnormal S-nitrosothiol levels have been measured ex vivo to monitor or diagnose a variety of human diseases or conditions, ranging, for example, from altitude acclimatization to asthma, pulmonary hypertension and sepsis. But S-nitrosothiols are labile and can be lost during sample extraction and processing. A need exists for technology to make clinically reliable S-nitrosothiol measurements in living tissues in situ.

Presently available methods to measure S-nitrosothiols do not satisfy this need and have other limitations. Methods involving mass spectrometry require inert liquid chromatography to separate S-nitrosothiols while preserving the delicate S—NO bond. The limit of sensitivity of colorimetric assays is generally out of range for many or most biological S-nitrosothiols. Mass spectrometric methods provide specificity but they are not suitable for liquid phase in-vivo measurements. Fluorescent labels can be used for identifying S—NO bonds but, among other limitations, the fluorescent dyes are not approved for clinical use in human medicine. Antibody labeling is also possible but it requires harsh conditions. Moreover, the specificity of the available antibodies is not consistent.

Chemiluminescence-based assays are the current standard for measuring S-nitrosothiols. In general terms, these assays use photolysis or chemical reduction of the S—N═O functional groups in S-nitrosothiols to produce nitric oxide (NO). The NO is then mixed with ozone to generate NO₂*, which decays back to NO₂, releasing one photon per molecule of NO. The photons are quantitated using a photomultiplier tube. Both photolytic and reductive chemiluminescence techniques are specific and sensitive.

The main limitation of all such chemiluminescence-based methods is that they require removing a tissue or blood sample, which is then treated with radiation or chemicals before a concentration can be determined. The current methods are also time-consuming and complicated. Moreover, due to their labile nature, S-nitrosothiols decay rapidly when removed from the body, and must therefore be measured quickly for an accurate reading.

Thus, a need exists for a faster, more efficient, more physiologically relevant, and less invasive system and method that allows scientists and medical professionals to measure S-nitrosothiols as a way to detect early signs of disease. The present disclosure addresses this need.

### SUMMARY

The present disclosure relates to systems and methods for determining the levels of S-nitrosothiols that can be used/performed in situ in a mammal without removing a biological sample from the mammal. In accordance with certain aspects of the disclosure, a target site in the body of a mammal is exposed transiently (for example, for approximately 10 seconds) to ultraviolet light, which cleaves the S—N bond in S-nitrosothiols, releasing nitric oxide. The released nitric oxide is measured to determine the S-nitrosothiol concentration. In various embodiments, the evolved nitric oxide is either directly measured (for example, by a nitric oxide analyzer) or is received in a reservoir (for example, a glass sampling syringe) to be transported for measurement, such as, for example, in a remote laboratory.

In an aspect, the present disclosure provides a system for determining levels of an S-nitrosothiol in situ by measuring nitric oxide released from a target site in the body of a mammal after exposure to ultraviolet light, comprising: a housing having a proximal end and a distal end and defining a cavity therebetween, the housing further defining an opening at the distal end, wherein the housing is configured for placement of the distal end in proximity with the target site to form a seal between the distal end of the housing and the target site, wherein a portion of the target site is exposed to the cavity through the opening;
a source of ultraviolet light affixed to the proximal end of the housing and positioned to irradiate the portion of the target site through the opening; and a nitric oxide analyzer configured to receive a gas sample from the cavity following irradiation of the target site and operable to measure a nitric oxide concentration in the gas sample.

In an embodiment of the system, the levels of the S-nitrosothiol in situ are determined based on a correlation with the nitric oxide concentration. In an embodiment, the target site is one or more of an organ, tissue, body cavity, and body fluid. In an embodiment, the organ or tissue is an organ or tissue having high vascularization.

In an embodiment, the target site is one or more of skin, oral cavity, and spinal fluid. In an embodiment, the skin is the skin of a finger or an ear.

In an embodiment, the system further comprises a lens movably affixed to the housing, the lens operable to focus the source of ultraviolet light upon the target site. The lens may be a double convex focusing lens having an effective focal length of from about 12 mm to about 36 mm and a diameter of from about 6 mm to about 18 mm.

In an embodiment, the ultraviolet light comprises light having a wavelength of from about 300 to about 375 nm.

In an embodiment, the nitric oxide analyzer performs a chemiluminescence-based assay.

In an embodiment, the S-nitrosothiol is S-nitroso-L-cysteine or S-nitrosoglutathione.

In an embodiment, the system is operable to diagnose or monitor a disease or condition. In an embodiment, the disease or condition is sepsis. In an embodiment, the system is further operable to diagnostically exclude that the disease or condition is cardiogenic shock.

In another aspect, the present disclosure provides a method of determining levels of an S-nitrosothiol in situ by measuring nitric oxide released from a target site in the body of a mammal after exposure to ultraviolet light, comprising: irradiating the target site with ultraviolet light to generate nitric oxide from the S-nitrosothiol; receiving the nitric oxide; and measuring the nitric oxide. In an embodiment of the method, the levels of the S-nitrosothiol in situ are determined based on a correlation with the nitric oxide concentration.

In an embodiment, the irradiating with ultraviolet light involves focusing the ultraviolet light using a lens. In an embodiment, the lens is a double convex focusing lens effective focal length of from about 12 mm to about 36 mm and a diameter of from about 6 mm to about 18 mm. In an embodiment, the ultraviolet light comprises light having a wavelength of from about 300 to about 375 nm. In an embodiment, the irradiating with the ultraviolet light the target site is performed for a duration of from about 1 to about 30 seconds.

In an embodiment of the method, the target site is one or more of skin, oral cavity, and spinal fluid.

In an embodiment, the measuring the nitric oxide is performed by a nitric oxide analyzer.

In an embodiment of the method, determining the levels of the S-nitrosothiol is performed to diagnose or monitor a disease or condition. In an embodiment, the disease or condition is sepsis.

In still another aspect, the present disclosure provides a method of distinguishing sepsis from cardiogenic shock in a patient in need thereof, the method comprising: determining levels of S-nitrosothiol in situ by measuring nitric oxide released from a target site in the patient's body after exposure to ultraviolet light, comprising: irradiating the target site with ultraviolet light to generate nitric oxide from the S-nitrosothiol, receiving the nitric oxide, and measuring the nitric oxide concentration; and based on the nitric oxide concentration measurement, determining whether the patient has sepsis of cardiogenic shock.

Further features, characteristics and embodiments of the present disclosure will be apparent from the detailed description herein.

### BRIEF DESCRIPTION OF THE DRAWINGS

FIG. 1 shows an illustrative arrangement of the elements of a system for determining levels of S-nitrosothiols in a mammal.

FIGS. 2A-2B are representations of an exemplary embodiment of the system of the present disclosure. FIG. 2A is a photograph showing the housing of the system of the present disclosure with its distal end placed in proximity with a well plate during the in vitro tests described in the Examples. FIG. 2B is a line drawing depicting the set-up of the embodiment shown in the photograph of FIG. 2A.

FIG. 3 is a photograph showing the housing of the system of the present disclosure as described above with its distal end placed in proximity with a rat ear during the in vivo tests described in the Examples.

FIGS. 4A-4C are plots of the nitric oxide released for each of the concentrations of GNSO in the in vitro tests described in the Examples. FIG. 4A displays NO release for 200, 100, and 50 micromolar S-nitrosoglutathione (GSNO). FIG. 4B displays NO release for 10, 5, and 1 micromolar GSNO. The peaks in FIG. 4A represent the three most concentrated standards (200, 100, and 50 μM). Each was done in duplicate, so the first two peaks (from the left) are from a 200 μM standard, the next two are from 100 μM standards, etc. Three different concentrations are shown in FIG. 4B: 10, 5, and 1 μM (the first very large spike on the left is 50 μM). FIG. 4C is the standard curve obtained by plotting the average nitric oxide values against the initial GSNO concentrations.

FIG. 5 shows a plot of the average nitric oxide released (mv*min) for each of eight rats used in the in vivo trial studies 1 and 2 described in the Examples.

FIGS. 6A-6B show plots of the nitric oxide released in parts per billion (ppb) for the ear (FIG. 6A) and the forearm (UV on; UV off; ambient) (FIG. 6B) in the human trial studies described in the Examples.

FIG. 7 shows a plot of the photoablation study performed as part of the human trials described in the Examples.

FIGS. 8A-8B are graphs depicting data from the sepsis experiments described in the Examples. FIG. 8A shows photolytic measurement readings obtained at the wrist. Left (dark grey) bar depicts control patients. Right (light grey) bar depicts patients with septic shock. Y-axis is nitric oxide (NO) measured by device in parts per billion. Patients with septic shock had significantly higher levels of NO measured by device when compared to controls (p-value=0.0196). FIG. 8B is a graphical representation of photolytic measurements obtained during shock (left) and after resolution of shock (right). Photolytic measurements obtained after resolution were statistically significantly lower than the measurements obtained during shock state.

FIG. 9 shows a receiver operator characteristic (ROC) curve derived from S-nitrosothiol measurements from the sepsis experiments according to the Examples. Area under the receiver operator characteristic curves are denoted in the bottom left (AUC=0.7567).

### DETAILED DESCRIPTION

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “another embodiment,” “some embodiments,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should be further appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

Throughout this disclosure, various quantities, such as amounts, sizes, dimensions, proportions and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The disclosed embodiments may, in some cases, be implemented in hardware, firmware, software, or a combination thereof. The disclosed embodiments may also be implemented as instructions carried by or stored on one or more transitory or non-transitory machine-readable (e.g., computer-readable) storage media, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may not be included or may be combined with other features.

According to one aspect of the present disclosure, a system is provided that includes a housing, a source of ultraviolet light and a nitric oxide analyzer. In one embodiment, the housing has a proximal end and a distal end and defines a cavity therebetween. In an embodiment, the housing further defines an opening at the distal end and is configured for placement of the distal end in proximity with a target site in the body of a mammal to form a seal between the distal end of the housing and the target site, with a portion of the target site exposed to the cavity through the opening. In an embodiment, the source of ultraviolet light is affixed to the proximal end of the housing and positioned to irradiate the portion of the target site through the opening. In an embodiment, the nitric oxide analyzer is configured to receive a gas sample from the cavity following irradiation of the target site and is operable to measure a nitric oxide concentration in the gas sample.

According to one aspect of the present disclosure, the system of the present disclosure is operable to determine levels of S-nitrosothiols in situ by measuring nitric oxide released from a target site in the body of a mammal after exposure to ultraviolet light.

In one embodiment, the system integrates the source of ultraviolet light with the nitric oxide analyzer. For example, in certain embodiments, the source of ultraviolet light, and optionally the nitric oxide analyzer, are positioned to interface with the target site in the body of a mammal.

In one embodiment of the system, the levels of S-nitrosothiols in situ can be inferred or calculated based on the measured nitric oxide values, e.g., by correlating the measured nitric oxide values to in vivo S-nitrosothiol levels that correspond to the measured nitric oxide values.

According to another aspect of the present disclosure, a method is provided for determining levels of S-nitrosothiols in situ by measuring nitric oxide released from a target site in the body of a mammal after exposure to ultraviolet light. In one embodiment, the method includes irradiating the target site with ultraviolet light to generate nitric oxide from the S-nitrosothiol; receiving the nitric oxide; and measuring the nitric oxide.

In certain embodiments, the method uses ultraviolet light in conjunction with a nitric oxide analyzer to cleave S-nitrosothiols in a target site in the body of a mammal, measure the released nitric oxide, and determine the corresponding levels of S-nitrosothiols in a target site in the body of a mammal without excising or extracting a tissue or other biological sample. In one embodiment, the method uses concentrated ultraviolet light to irradiate the target site.

The method according to one embodiment uses a source of ultraviolet light integrated with the nitric oxide analyzer, wherein the source of ultraviolet light, and optionally the nitric oxide analyzer, interface with the target site in the body of a mammal. The target site is irradiated with the ultraviolet light, which cleaves the sulfur-nitrogen bonds in S-nitrosothiols, releasing nitric oxide. The system then directly measures the released nitric oxide. The method is non-invasive in that it does not involve removing a biological sample from the mammal's body.

In one embodiment of the method, the levels of S-nitrosothiols in situ are inferred or calculated based on the measured nitric oxide values, e.g., by correlating the measured nitric oxide values to in vivo S-nitrosothiol levels that correspond to the measured nitric oxide values.

In one embodiment, measurement of nitric oxide is performed using a chemiluminescence-based assay. These assays use photolysis or chemical reduction of the S—N═O functional groups in S-nitrosothiols to produce nitric oxide (NO). The NO is then mixed with ozone to generate NO₂*, which decays back to NO₂, releasing one photon per molecule of NO. The photons are quantitated using a photomultiplier tube.

In one embodiment, the nitric oxide is received in a reservoir (for example, a glass sampling syringe) to be transported for measurement, such as, for example, in a separate instrument and/or in a remote laboratory.

In one embodiment, the method involves receiving and measuring the released nitric oxide by a nitric oxide analyzer, such as, for example, the Sievers Nitric Oxide Analyzer NOA 280i obtainable from Zysense, LLC, which has a registered office in North Carolina.

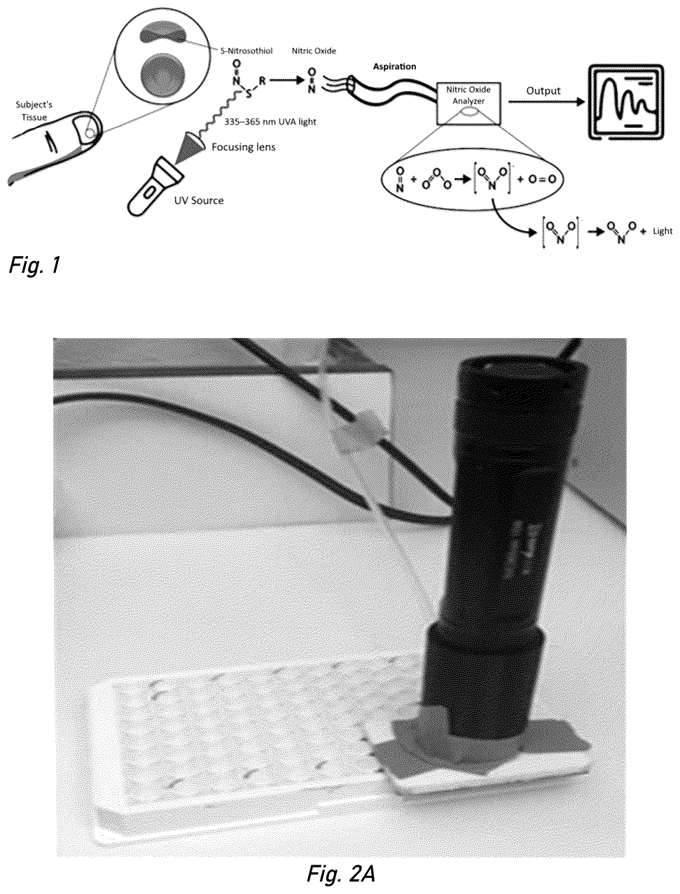

FIG. 1 shows an illustrative arrangement of the system and/or method of the present disclosure. In the embodiment shown, a source of ultraviolet light is placed in proximity with the target site (such as, for example, a finger). The ultraviolet light is focused with the help of a lens. Exposure to the focused UV light (shown by wavy purple lines) cleaves the S—N bond in the S-nitrosothiols and releases nitric oxide (NO). The released nitric oxide is aspirated into a nitric oxide analyzer (e.g., NOA 280i), as shown by dark wavy lines. This is represented by the wavy line and the first chemical equation in FIG. 1. The nitrous oxide analyzer (e.g., NOA 280i) then measures the nitric oxide content of the sample.

As will be appreciated from the descriptions herein, a wide variety of aspects and embodiments are contemplated by the present disclosure, examples of which include, without limitation, the aspects and embodiments listed below:

A system for determining levels of an S-nitrosothiol in situ that includes: (i) a housing having a proximal end and a distal end and defining a cavity therebetween, the housing further defining an opening at the distal end, wherein the housing is configured for placement of the distal end in proximity with a target site in the body of a mammal to form a seal between the distal end of the housing and the target site, wherein a portion of the target site is exposed to the cavity through the opening; (ii) a source of ultraviolet light affixed to the proximal end of the housing and positioned to irradiate the portion of the target site through the opening; and (iii) a nitric oxide analyzer configured to receive a gas sample from the cavity following irradiation of the target site and operable to measure a nitric oxide concentration in the gas sample.

A system in accordance with any other embodiment disclosed herein, wherein the seal that is formed between the distal end of the housing and the target site is a seal that retains enough of the nitric oxide in the cavity such that the levels of the S-nitrosothiols can be reliably determined in situ.

A system in accordance with any other embodiment disclosed herein, wherein the target site is one or more of an organ, tissue, body cavity, or body fluid.

A system in accordance with any other embodiment disclosed herein, wherein the system is used in conjunction with another system such as, for example, an endoscope, a bronchoscope, a cystoscopy device, an intracranial monitor, and intra-amniotic monitor and/or a catheter such as an intravascular catheter, urinary catheter or other catheter.

A system in accordance with any other embodiment disclosed herein, wherein the target site is one or more of skin, oral cavity, and spinal fluid.

A system in accordance with any other embodiment disclosed herein, further comprising a lens movably affixed to the housing, the lens operable to focus the source of ultraviolet light upon the target site.

A system in accordance with any other embodiment disclosed herein, wherein the lens is a double convex focusing lens.

A system in accordance with any other embodiment disclosed herein, wherein the lens is a double convex focusing lens having an effective focal length of from about 12 mm to about 36 mm and a diameter of from about 6 mm to about 18 mm.

A system in accordance with any other embodiment disclosed herein, wherein the lens is a double convex focusing lens having an effective focal length of from about 18 mm to about 30 mm and a diameter of from about 9 mm to about 15 mm.

A system in accordance with any other embodiment disclosed herein, wherein the lens is a double convex focusing lens having an effective focal length of about 24 mm and a diameter of about 12 mm.

A system in accordance with any other embodiment disclosed herein, wherein the ultraviolet light comprises light having a wavelength of from about 10 to about 400 nm, from about 50 to about 390 nm, from about 100 to about 385 nm, from about 150 to about 380 nm, from about 200 to about 375 nm, from about 250 to about 375 nm, from about 300 to about 375 nm, from about 350 to about 375 nm, from about 360 to about 375 nm, or about 365 nm.

A system in accordance with any other embodiment disclosed herein, wherein the nitric oxide analyzer performs a chemiluminescence-based assay.

A system in accordance with any other embodiment disclosed herein, wherein the chemiluminescence-based assay involves photolytic chemiluminescence, reductive chemiluminescence or both.

A system in accordance with any other embodiment disclosed herein, wherein the S-nitrosothiol is S-nitroso-L-cysteine or S-nitrosoglutathione.

A system in accordance with any other embodiment disclosed herein, wherein the target site is an organ or tissue having high vascularization.

A system in accordance with any other embodiment disclosed herein, wherein the organ or tissue is skin.

A system in accordance with any other embodiment disclosed herein, wherein the skin is the skin of a finger or an ear.

A system in accordance with any other embodiment disclosed herein, wherein the mammal is a living mammal.

A system in accordance with any other embodiment disclosed herein, wherein the mammal is a human patient. In such embodiments, the human patient may be a pediatric patient.

A system in accordance with any other embodiment disclosed herein, which is operable to diagnose or monitor a disease or condition.

A system in accordance with any other embodiment disclosed herein, which is operable to diagnose or monitor one or more of asthma, pulmonary hypertension, and sepsis.

A system in accordance with any other embodiment disclosed herein, which is operable to diagnose or monitor sepsis. The system may be further operable to diagnostically exclude that the disease or condition is cardiogenic shock. In such embodiments, a nitrous oxide concentration ≥1 ppb may be correlated with a positive diagnosis for sepsis.

A method of determining levels of an S-nitrosothiol in situ that includes: (i) irradiating with ultraviolet light a target site in the body of a mammal to generate nitric oxide from the S-nitrosothiol; (ii) receiving the nitric oxide; and (iii) measuring the nitric oxide. In any embodiment, measuring the nitric oxide may comprise measuring the concentration of nitric oxide.

A method in accordance with any other embodiment disclosed herein, wherein the irradiating with ultraviolet light involves focusing the ultraviolet light using a lens.

A method in accordance with any other embodiment disclosed herein, wherein the lens is a double convex focusing lens having an effective focal length of from about 12 mm to about 36 mm and a diameter of from about 6 mm to about 18 mm.

A method in accordance with any other embodiment disclosed herein, wherein the ultraviolet light comprises light having a wavelength of from about 10 to about 400 nm, from about 50 to about 390 nm, from about 100 to about 385 nm, from about 150 to about 380 nm, from about 200 to about 375 nm, from about 250 to about 375 nm, from about 300 to about 375 nm, from about 350 to about 375 nm, from about 360 to about 375 nm, or about 365 nm.

A method in accordance with any other embodiment disclosed herein, wherein the target site is one or more of an organ, tissue, body cavity, or body fluid.

A method in accordance with any other embodiment disclosed herein, wherein the target site is one or more of skin, oral cavity, and spinal fluid.

A method in accordance with any other embodiment disclosed herein, wherein the irradiating with the ultraviolet light the target site is performed for a duration of from less than one second to about 30 seconds, from about 1 to about 30 seconds, from about 2 to about 25 seconds, from about 3 to about 20 seconds, from about 4 to about 15 seconds, from about 5 to about 15 seconds, or from about 6 to about 10 seconds.

A method in accordance with any other embodiment disclosed herein, wherein the measuring the nitric oxide comprises performing a chemiluminescence-based assay.

A method in accordance with any other embodiment disclosed herein, wherein the chemiluminescence-based assay involves photolytic chemiluminescence, reductive chemiluminescence or both.

A method in accordance with any other embodiment disclosed herein, wherein the measuring the nitric oxide is performed by a nitric oxide analyzer. In such embodiments, the nitric oxide analyzer may be an ozone-chemiluminescence nitric oxide analyzer (e.g., Zysense NOA 280i or EcoPhysics nCLD 84M).

A method in accordance with any other embodiment disclosed herein, wherein the mammal is a living mammal.

A method in accordance with any other embodiment disclosed herein, wherein the mammal is a human patient. The human patient may be a pediatric patient.

A method in accordance with any other embodiment disclosed herein, wherein the method obviates removing a sample of a tissue or fluid from the mammal.

A method in accordance with any other embodiment disclosed herein, wherein the determination of the levels of the S-nitrosothiol is performed to diagnose or monitor a disease or condition.

A method in accordance with any other embodiment disclosed herein, wherein the disease or condition is one or more of asthma, pulmonary hypertension, and sepsis.

A method in accordance with any other embodiment disclosed herein, wherein the disease or condition is sepsis. The method may further comprise diagnostically excluding that the disease or condition is cardiogenic shock. In such embodiments, a nitrous oxide concentration ≥1 ppb may be correlated with a positive diagnosis for sepsis. In such embodiments, the method may further comprise: treating the mammal for sepsis if a nitrous oxide concentration reading is ≥1 ppb.

A method in accordance with any other embodiment disclosed herein, wherein the determination of the levels of the S-nitrosothiol is performed to achieve altitude acclimatization in a mammal.

A method in accordance with any other embodiment disclosed herein, wherein the determination of levels of the S-nitrosothiol is performed after a medical treatment intervention, in order to monitor the effects of the treatment intervention.

In another aspect, the present disclosure provides a method of monitoring the effects of a medical treatment in a patient in need thereof comprising: treating the patient, then performing the method of determining levels of an S-nitrosothiol in situ in according with any embodiment disclosed herein.

In a further aspect, the present disclosure provides a method of measuring capillary bed perfusion in a subject in need thereof, comprising: using the system according to any embodiment disclosed herein to measure S-nitrosothiol levels in the capillary bed of a tissue of the subject.

In an additional aspect, the present disclosure provides a method of monitoring tissue oxygenation in a subject in need thereof, comprising: using the system according to any embodiment disclosed herein to measure the subject's tissue S-nitrosothiol levels.

In still another aspect, the present disclosure provides a method of monitoring high-altitude acclimatization in a subject in need thereof, comprising: using the system according to any embodiment disclosed herein to measure a baseline level of the subject's tissue S-nitrosohaemoglobin levels circulating in the subject's blood; and performing one or more comparison measurements of the subject's tissue S-nitrosohaemoglobin levels circulating in the subject's blood.

In yet another aspect, the present disclosure provides a method of distinguishing sepsis from cardiogenic shock in a patient in need thereof, the method comprising: determining levels of S-nitrosothiol in situ by measuring nitric oxide released from a target site in the patient's body after exposure to ultraviolet light, comprising: irradiating the target site with ultraviolet light to generate nitric oxide from the S-nitrosothiol, receiving the nitric oxide, and measuring the nitric oxide concentration; and based on the nitric oxide concentration measurement, determining whether the patient has sepsis of cardiogenic shock. In any embodiment, the patient may be a pediatric patient.

In still another aspect, the present disclosure provide the use of a device for the manufacture of a system for diagnosis of sepsis, the device comprising a housing having a proximal end and a distal end and defining a cavity therebetween, the housing further defining an opening at the distal end, wherein the housing is configured for placement of the distal end in proximity with a target site in the body of a mammal to form a seal between the distal end of the housing and the target site, wherein a portion of the target site is exposed to the cavity through the opening; (ii) a source of ultraviolet light affixed to the proximal end of the housing and positioned to irradiate the portion of the target site through the opening; and (iii) a nitric oxide analyzer configured to receive a gas sample from the cavity following irradiation of the target site and operable to measure a nitric oxide concentration in the gas sample.

In another aspect, the present disclosure provides the use of a device for the manufacture of a system for distinguishing sepsis from cardiogenic shock in a patient in need thereof, the device comprising a housing having a proximal end and a distal end and defining a cavity therebetween, the housing further defining an opening at the distal end, wherein the housing is configured for placement of the distal end in proximity with a target site in the body of a mammal to form a seal between the distal end of the housing and the target site, wherein a portion of the target site is exposed to the cavity through the opening; (ii) a source of ultraviolet light affixed to the proximal end of the housing and positioned to irradiate the portion of the target site through the opening; and (iii) a nitric oxide analyzer configured to receive a gas sample from the cavity following irradiation of the target site and operable to measure a nitric oxide concentration in the gas sample.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Examples

Examples related to the present disclosure are described below. In some embodiments, alternative techniques can be used. The examples are intended to be illustrative and are not limiting or restrictive of the scope of the invention as set forth in the claims. Changes therein and other uses which are encompassed within the spirit of the invention as defined by the scope of the claims will occur to those of ordinary skill in the art.

### Example 1. System and Method for Measuring S-Nitrosothiols In Situ

As detailed in this section, the system and method of the present disclosure was subject to both in vitro tests and in vivo trials. The in vitro tests validated that the UV light and gas inlet systems worked together to stimulate and measure the production of nitric oxide. The in vitro tests also established a correlation between the output values from the nitric oxide analyzer, NOA 280i, with chemical standards of known concentrations of S-nitrosoglutathione (GSNO) to form a standard curve. The in vivo trials showed that the system and/or method is operable to detect nitric oxide released in animal subjects upon exposure of the ear to ultraviolet light.

### Materials and Methods

### Instrumentation.

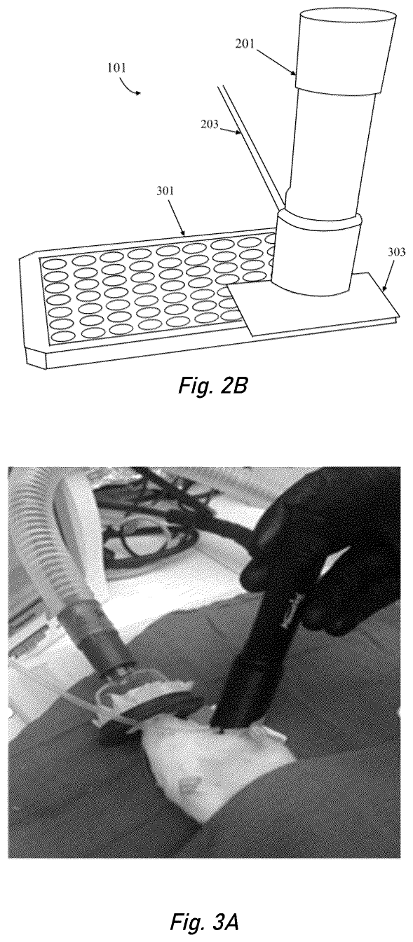

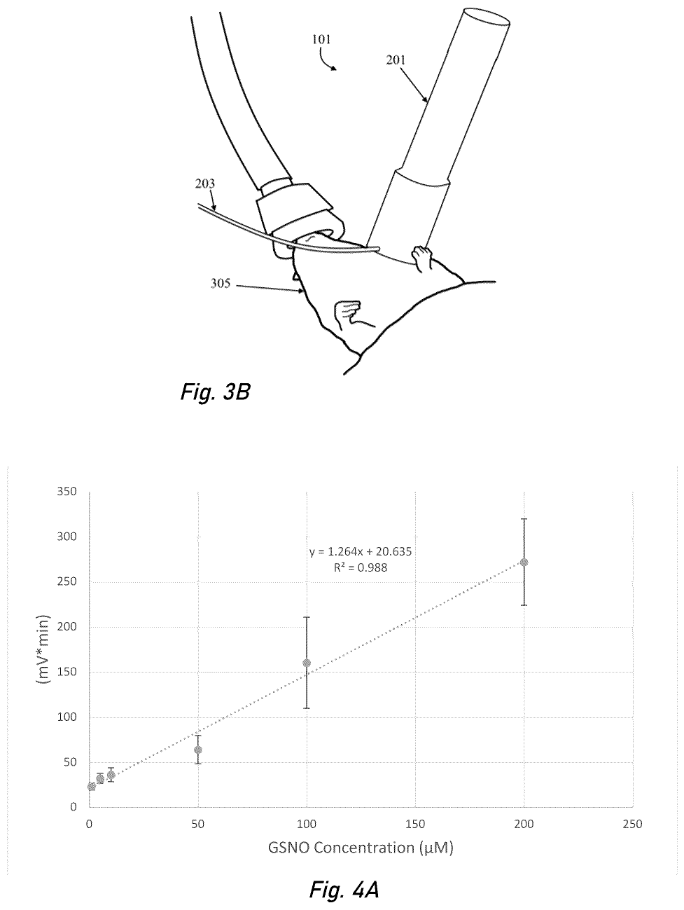

With reference to FIG. 2B and FIG. 3B, the system 101 included a device 201 comprising of a UV light source and focusing lens, with a custom tissue interface and having a port for tubing 203 for gas analysis. In the embodiment used in the present example, the device 201 is a UV light source housed in a custom-machined tissue interface with a side port for gas analysis. More specifically, the UV light source used is an SV003 10W UV light device (Alonefire, Shenzhen City, China) which produced UV light at a 365 nm wavelength. Light was focused using a 12 mm diameter and 24 mm effective focal length double convex focusing lens (Edmund Optics, Barrington, NJ/USA) used to concentrate the ultraviolet light. The luminous power was 2,400 mW, and the intensity in the area of focused exposure (0.5 cm radius) was 1.88 W/cm². A custom 3D print 303 was created to hold the focusing lens in proximity with the well plate 301 for the in vitro experiments, and hold the suction tube 203 in place (the suction tube being used to transport the nitric oxide gas to the nitric oxide analyzer). This was printed on a Fortus® 400mc (Stratasys, Eden Prairie, MN/USA) using 0.330 mm slice layer thickness and 100% infill. A machined piece was used to interface with the well plate 301 or subject rat's ear 305. This device was connected by a side port and Teflon tubing 203 to one of two NO analyzers (not shown in FIG. 2B or FIG. 3B), each calibrated according to the manufacturers' instructions. This UV system was used to cleave the bond in the S-nitrosothiol and release the nitric oxide. The Sievers Nitric Oxide Analyzer 280i, NOA 280i (Zysense, North Carolina, USA) was used as the nitric oxide analyzer.

### S-Nitrosoglutathione.

Lyophilized S-nitrosoglutathione (GSNO) was made by a partner lab and dissolved in pure water from a Milli-Q® ultrapure water system (MilliporeSigma, Burlington, MA/USA). Upon receiving the lyophilized GSNO, a 100 mM stock solution was created by dissolving 0.0554 g of powder in 1.5 mL of pure water (accounting for the 91% purity of GSNO powder). After the creation of the stock solution, serial dilutions were used to create standard solutions that spanned a wide range, including the expected biological range of SNO concentrations. The concentrations of the standard solutions used were 200, 100, 50, 10, 5, and 1 μM GSNO. For the in-vitro tests, the standards were pipetted into 96-well opaque polystyrene microplates that prevented the passage of UV light from one well to another (Corning Incorporated, Corning, NY/USA).

### In-Vitro Tests.

Photolysis of the samples was achieved by placing the source of ultraviolet light and the lens about 2 centimeters from the top of the individual well being tested, as shown in FIGS. 2A-2B.

Released nitric oxide was aspirated into the NOA 280i nitrous oxide analyzer (not shown in FIGS. 2A-2B) at the intake flow rate of the instrument Teflon tubing 203. Standards were pipetted into 96-well opaque polystyrene microplates 301 that prevented the passage of UV light from one well to another (Corning Incorporated, Corning, NY, USA). Head space NO was aspirated into the NOA 280i at the intake flow rate of the Sievers instrument (200 mL/min).

Testing was done to determine the optimum UV exposure times for the samples in order to find the shortest UV exposure time that still yielded the maximum amount of nitric oxide release. The experiment consisted of nitric oxide release as the independent variable and UV exposure time as the dependent variable. The concentrations of GSNO used for the standard solutions were 200, 100, 50, 10, 5, and 1 μM. There were 8 experimental groups that consisted of 200 μL of each standard exposed to 6, 8, 10, 12, 14, and 16 seconds of UV light. The 2 additional groups were controls; negative control 1 had no GSNO and negative control 2 had no UV light. Each group was tested five times. An exposure time of 10 seconds was chosen for standard curve creation and animal testing.

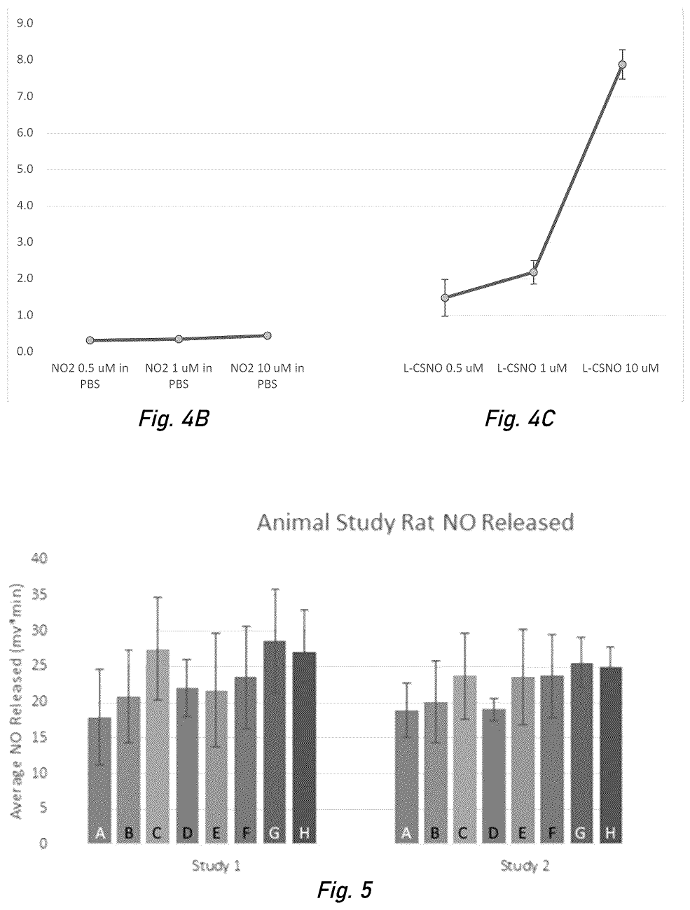

To create the standard curve, each GSNO standard was tested three times. The photolyzed GSNO released nitric oxide and caused the machine to display a response value in software that was processed and matched with the concentration of the standard. In this way, a standard curve plotting GSNO concentration against total nitric oxide collected was created (FIG. 4A). The photolyzed GSNO released NO and caused the machine to display a response value in nV and mV*min. When exposed to the photolytic system, nitrite in PBS evolved ˜ 1/100^{th} the NO that was evolved from S-nitrosothiol in PBS (FIGS. 4B-4C). Nitrate produced no NO photolytically.

### In-Vivo Trials.

Eight Sprague Dawley rats (four male and four female, designated rats A, B, C, D, E, F, G, and H) were used for the in-vivo trials. Rats were fed a standard diet. Weights ranged from −250-500 g, with a length of exposure time of 10 seconds. The target location in this study was the ear (FIGS. 3A-3B) due to its relative transparency and ease of access. The assay was performed while the rat was under isoflurane gas anesthesia so that changes in the animals' position were reduced. Rats were anesthetized using isoflurane induction via a rodent induction chamber (isoflurane 1-5% in oxygen, 0.5-2 L/min flow rate). This also slowed blood flow, allowing for less blood to be exposed to a greater amount of UV light. The ultraviolet light source and gas intake device were placed about 2 millimeters from the rat's ear. This distance was the same distance as the placement of the device relative to the in-vitro GSNO solution's gas-permeable membrane. Then, using the correlation between the mV output from the NOA 280i and NO concentration from the in-vitro study, the concentration of the S-nitrosothiols was determined.

### Human Studies.

The procedure for the assay in normal, healthy human subjects was similar to that for the rat study, with the following modifications: First, anesthesia was not required. Second, a maximum exposure time of 40 seconds was used to prevent any chance of UV burn. Third, it was determined that the ear lobe or the ventral forearm provided the best interface for the light/sampling device. Fourth, the subject breathed through a mouthpiece while wearing nose clips to prevent side stream intake of exhaled NO into the device when assays were done on the ear. Finally, though the Zysense NOA was used initially, the time axis proved to be more precisely visualized using the EcoPhysics NOA (Ann Arbor, Michigan, USA). After further testing of a variety of anatomical sites for the assay, it was determined that a variety of anatomical sites are suitable, including various sites from neck to leg. One suitable anatomical site that was identified through optimization studies is the shoulder.

### Statistics.

ANOVA and subsequent Tukey comparison were performed at a significance level of 0.05 to determine the optimal UV exposure time. A Fisher comparison of the same data was also performed. A t-test was performed at a significance level of 0.01 to compare NO release from the ex-vivo samples treated and untreated with GSNO.

### Results

### In-Vitro Tests.

The ANOVA and Tukey comparison indicated that all exposure times were the same. The Fisher comparison indicated that the shortest times, 2 and 4 seconds, had significantly lower nitric oxide release than all the other times. The second ANOVA and Tukey comparison indicated that there were no significant differences between any of the times, possibly indicating that exposure times as low as 6 seconds were capable of photolyzing entire samples. However, because 10 seconds yielded the best average response in initial tests, it was selected as the time for further experimentation.

After determining that 10 seconds was the optimal ultraviolet exposure time, a standard curve was created. The data gathered from the in-vitro studies consisted of a list of mv*min values given by NOA 280i analysis software after reading the appropriate standards. The system was able to detect NO from all photolyzed standard solutions. FIGS. 4A-4C display various outputs for the different concentration. The R² was 0.988.

Plotting the average nitric oxide values against the initial GSNO concentrations yielded a standard curve, shown in FIG. 4A. The curve displays a strong linear relationship, with a high coefficient of determination of 0.988. The associated equation (shown below) was used to correlate nitric oxide release with known concentrations and eventually determine the S-nitrosothiol content of biological samples with unknown concentrations.

$$y = 1.264 x + 20.635$$

In the above equation, “y” is NO released, and “x” is the initial S-nitrosothiol concentration.

NO release was measured from NaNO₂ and L-CSNO dissolved in PBS. As shown in FIG. 4B, NO evolved from increasing concentrations of NaNO₂ was between 0.3 and 0.4 ppb, which is equal to ambient air, while, as shown in FIG. 4C, L-CSNO gave proportional increase of NO signaling (from 1.5 to 7.9 ppb) with its increased concentration.

### In-Vivo Animal Trials.

The in vivo animal studies were broken down into two trials, with the first trial determining the location of photolytic cleavage and confirming the in vitro values for the duration of the light exposure. Three replicates of ultraviolet light exposure were performed.

Eight rats (four males; four females, designated A, B, C, D, E, F, G, and H) were subject to a total of six periods of ultraviolet light exposure, each period being of the duration of 10 seconds. A plot of the resulting averages is shown FIG. 5. There was also a negative control performed and recorded (UV light off).

There was clear statistical difference between the rat readings and the negative control (UV light off), indicating that the UV light is cleaving one or more S-nitrosothiols. Utilizing the standard curve from the in vitro studies, the average mv*min value can be converted into the μM S-nitrosothiol concentration.

The trial was then repeated one week later, and the results of that trial are shown below in Table 1:

TABLE 1

| | | Calculated tissue | | Calculated tissue |
|---|---|---|---|---|
| Sub- | mv*min | S-nitrosothiol | mv*min | S-nitrosothiol |
| ject | reading | Concentration (μM) | reading | Concentration (μM) |
| Rat A | 17.87 | Below detection limit | 18.85 | Below detection limit |
| Rat B | 20.77 | 0.10 | 20.07 | Below detection limit |
| Rat C | 27.43 | 5.38 | 23.63 | 2.37 |
| Rat D | 22.03 | 1.11 | 18.97 | Below detection limit |
| Rat E | 21.60 | 0.76 | 23.53 | 2.29 |
| Rat F | 23.42 | 2.20 | 23.62 | 2.36 |
| Rat G | 28.50 | 6.22 | 25.53 | 3.88 |
| Rat H | 26.98 | 5.02 | 24.92 | 3.39 |

The above S-nitrosothiol values do not exceed what would be expected in a living system. Most of the values in the table fit within the physiological range. Some of the rats had readings low enough that they could not be properly translated into ρM outputs. This does not mean there was no signal; just that the standard curve does not handle such small concentrations well.

The results of the paired t-test for the two trials indicated that the differences to be considered were not statistically significant, and that there was not considerable variation between the two trials. The test was at 95% confidence and the resulting p-value was 0.274.

### Human Studies.

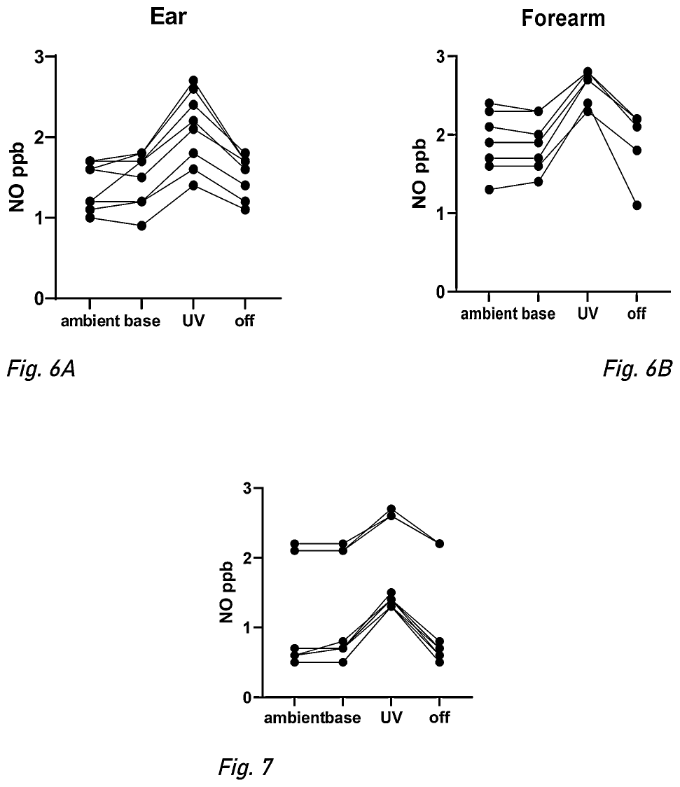

Four heathy individuals volunteered for this study. Nine total measurements were recorded from 3 ears (repeated 3 times) and 4 from the distal ventral forearm just proximal to the wrist. The UV light (or no-UV control) was placed for 30 second, FIGS. 6A-6B. The signal from the ear lobe (FIG. 6A) was consistently greater with the UV light on (UV) (2.1±0.5 ppb) than with the light off (1.5±0.3 ppb; N=9 studies from 3 subjects; p=0.0004). Likewise, the signal from the forearm (FIG. 6B) was consistently greater with the UV light on (UV) (2.6±0.3 ppb) than with the light off (1.8±0.5 ppb; N=7 studies from 4 subjects; p=0.023).

Photoablation did not change NO. Human forearms were repeatedly exposed to UV light as described above (n=11 from 4 subjects), with 20-second intervals between exposures. There was no diminishing NO signaling after three subsequent UV light exposures. Ambient NO was recorded for 10 seconds. Baseline NO (with probe put on the skin)—10 seconds, UV on was 40 seconds, UV off (time intervals between each measure) was 15-20 seconds 3 times made a difference in the NO signal (n=3 each, p=NS) (FIG. 7). This latter result differed from the rat ear study, in which there was evidence for photoablation.

Using the device described in the disclosure, data were also obtained data showing that children with sepsis admitted to the pediatric ICU have higher photolyzable S-nitrosothiol levels than control children without shock or with low-output (cardiogenic) shock. The clinical site of assay was also validated. After testing the assay at a variety of sites, a preferred site for the assay is the shoulder, although a variety of other sites such as leg and neck are also suitable.

### Example 2. In-Vivo Analysis of Tissue S-Nitrosothiols in Cases of Sepsis

Sepsis is a leading cause of morbidity and mortality worldwide. Globally there are an estimated 8 million deaths from sepsis. Sepsis is one of the leading causes of death in the pediatric population, over one third of global deaths from sepsis occur in children. Vascular dysfunction is common in sepsis and is a large driver of both mortality and development of multiple organ dysfunction syndrome. Nitrosative stress has been implicated in the pathophysiology of sepsis, and increased nitrosative stress has been associated with both severity of sepsis and organ dysfunction. Despite this, various therapies targeting nitrosative stress pathways have failed to improve outcomes in sepsis. Nitric oxide's role in sepsis is complex and has both favorable and unfavorable actions. A more complete understanding of nitrosative stress in sepsis is necessary to better tailor therapies.

S-nitrosothiols are involved in numerous physiologic pathways including blood pressure regulation, regulation of respiratory drive, and airway smooth muscle tone. Low-mass S-nitrosothiols are important signaling molecules that are predominantly produced by metalloproteins such as ceruloplasmin, hemoglobin, and NO synthase. Importantly these low-mass S-nitrosothiols have cyclic guanosine monophosphate (cGMP) independent bioactivities. Previously, S-nitrosothiol levels have been measured ex-vivo and found to be elevated in adult patients with sepsis. While these compounds have been implicated in many disease processes including sepsis, they are labile; thus measurement is difficult and currently limits its clinical utility. Recently, the present investigators designed and validated a novel approach to measure S-nitrosothiols in vivo. This device uses ultraviolet light homolytically to break the S-nitrosothiol bond, producing NO. NO can diffuse through the skin and be measured using an NO analyzer. Both sensitivity and specificity of this device was previously demonstrated: it measures nanoscopic quantities and concentrations of endogenous S-nitrosothiols, such as S-nitrosocysteine and S-nitrosoglutathione, and does not detect either nitrite or nitrate at physiological pH. This technology detects S-nitrosothiols in rats in vivo, e.g., as shown in Example 1, supra.

The present example demonstrates characterization of tissue S-nitrosothiol levels in-vivo in pediatric patients with sepsis. It was hypothesized that pediatric patients with sepsis would have higher levels of S-nitrosothiols as measured by the system of the present disclosure. It was also hypothesized that higher S-nitrosothiol levels would be associated with worse clinical outcomes. It was further hypothesized that higher S-nitrosothiol levels could distinguish sepsis from other conditions with similar presenting symptoms, such as cardiogenic shock.

### Materials and Methods

A prospective study of pediatric patients admitted to the pediatric intensive care unit was performed from 2021-2023. Patients with sepsis were eligible for enrollment if they were admitted to the intensive care unit with a diagnosis of sepsis and were on vasoactive support. Controls were primarily enrolled in the pediatric intensive care unit (PICU), and were patients without requiring any vasoactive support, an additional four patients without hemodynamic compromise were included from the pulmonary clinic. Patients were excluded if they had a chronic skin condition or burn excluding measurement of S-nitrosothiol levels, or if they were on inhaled NO therapy. This study was performed in accordance with Indiana University (IRB #10839). Informed consent was obtained by participants legal guardian prior to study enrollment. Assent was also obtained for patients aged 8-17 years unless their clinical situation precluded capacity for providing assent.

For S-nitrosothiol measurements, an embodiment of the system and method described herein was used. The specific embodiment used in the present example included: a UV light device (SV003 10W Alonefire, Shenzhen, China) which produces light at a 365 nm wavelength, as the UV source, and a 12 mm diameter double convex focusing lens with a 24 mm effective focal length. A custom 3D printed housing for the light, focusing lens, and side port tube was used. This device was then connected by a side port and Teflon tubing to an EcoPhysics NOA (Ann Arbor, MI, USA) to detect NO. A full measurement included a 10-second ambient air recording with the UV light off, a 10-second measurement with the device on the skin surface but with the light off, and a 10-second measurement with the device on the skin and the light on. Measurements were obtained in triplicate for each patient. Measurements were obtained at the palmar wrist, shoulder, and ear. To obtain final values reported, the ambient measurement was subtracted from the measurement obtained with device on the skin and with the UV light on. Significant exhaled breath contamination was noted for measurements obtained at shoulder and ear and therefore only wrist measurements are reported.

Patient demographics, laboratory data, PICU interventions, length of stay data, and medication data were collected from the electronic medical record. Pediatric Logistic Organ Dysfunction (PELOD) scores at the time of PICU admission were extracted from Virtual Pediatric Systems (VPS, LLC http://www.myvps.org/) database. Vasoactive inotrope scores were calculated at multiple time points. Development of acute respiratory distress syndrome (ARDS) was done using the Pediatric Acute Lung Injury Consensus Conference (PALICC-2) guidelines. Development of multiple organ dysfunction syndrome (MODS) was calculated using Goldstein's criteria. Both MODS and ARDS development were calculated in the first 24 hours and 72 hours after measurement of S-nitrosothiol levels. Exploratory outcomes included composite 28-day measures of the number of ventilator-free days, days free from vasopressor use, days alive and out of the ICU, and days alive and out of hospital.

Data were summarized and distributions were examined. Categorical variables were presented as counts and frequencies and were compared using x² or Fisher's exact as appropriate. Non-parametric continuous variables were presented as medians with interquartile ranges and compared with the Wilcoxon rank sum test. Univariate logistic and linear regressions were performed. To attempt to control for cohort type, a multivariate model was constructed using cohort type as a variable in the model. Paired T-tests were performed for paired measurements. An optimal cut-point was determined for S-nitrosothiol levels measured by the device using Liu's index. Receiver operator characteristic (ROC) curves were generated and the area under the ROC curve was denoted. A two-tailed p-value of <0.05 was considered statistically significant. Data were analyzed using STATA 17.

### Results

A total of 44 patients were enrolled with a median age of 7.5 years (IQR:2.5, 15). 23 cases were enrolled and 21 controls. There were three (6.8%) non-survivors in the cohort. The controls were younger [median age 5 years (IQR 0,9) versus 11 years (IQR: 6,16), p-value 0.012]. However, there was no relationship between age and S-nitrosothiol measurement r=0.28, p=0.060. The controls also had fewer comorbidities [7 (33.3%) versus 20 (87.0%), p-value <0.001] and lower PELOD scores [0 (IQR: 0,0) versus 12 (IQR: 11, 21), p-value <0.001]. There were no differences in gender, ethnicity, race, or body mass index (BMI) (see Table 2, below).

TABLE 2

| | Control | | Septic Shock | | p- |
|---|---|---|---|---|---|
| Variable | (n = 15) | | (n = 23) | | value |
| Age | 2 | (0, 7) | 11 | (6, 16) | <0.001 |
| BMI | 16.7 | (15.8, 18.9) | 18.1 | (14.5, 24.5) | 0.521 |
| Gender | | | | | 0.927 |
| Female | 5 | (33.3%) | 8 | (34.8%) | |
| Male | 10 | (66.7%) | 15 | (65.2%) | |
| Ethnicity | | | | | 0.999 |
| Hispanic | 0 | (0.0%) | 1 | (4.4%) | |
| Non-Hispanic | 15 | (100.0%) | 22 | (95.7%) | |
| Race | | | | | 0.999 |
| Asian | 0 | (0.0%) | 0 | (0.0%) | |
| Black | 1 | (6.7%) | 2 | (8.7%) | |
| Unknown | 0 | (0.0%) | 1 | (4.4%) | |
| White | 14 | (93.3%) | 20 | (87.0%) | |
| Presence of | 2 | (13.3%) | 20 | (87.0%) | <0.001 |
| Comorbidities | | | | | |
| PELOD¹ | 0 | (0, 1) | 12 | (11, 21) | <0.001 |

¹PELOD = Pediatric logistic organ dysfunction score

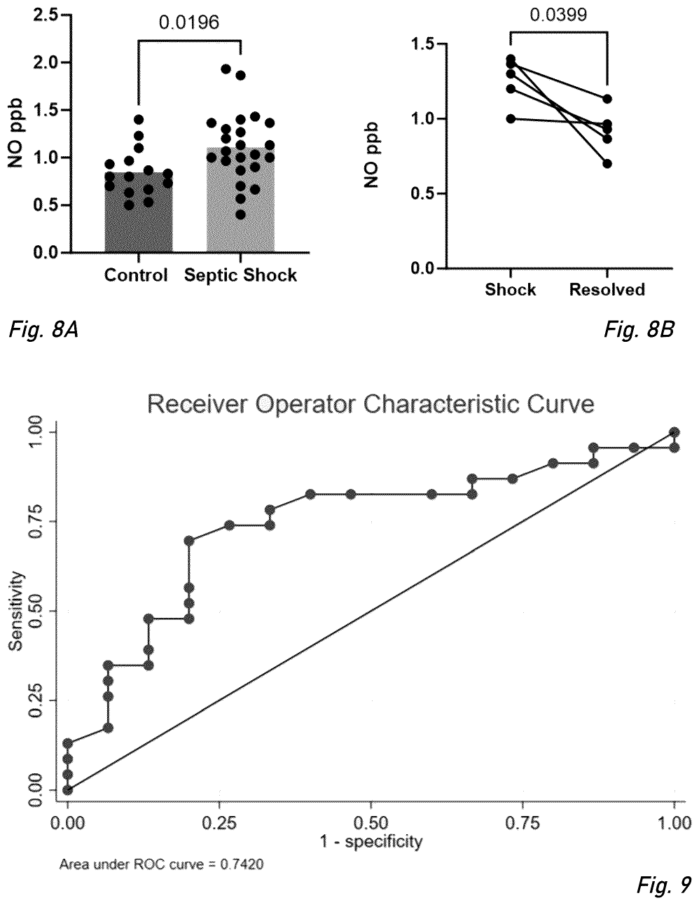

The primary outcome was difference in S-nitrosothiol levels as measured by the device between cohorts of patients. Patients with sepsis had statistically significantly higher NO measurements compared with controls [1.07 ppb (0.9, 1.4) versus 0.8 ppb (0.6, 0.97), p-value 0.004]. FIG. 8A denotes this difference. Five patients with sepsis have had repeat measures of S-nitrosothiol levels after resolution of shock. There is a significant decrease in S-nitrosothiol level after shock resolution [1.3±0.16 ppb versus 0.9±0.16 ppb, p-value 0.04], see FIG. 81B.

### Determination of Abnormal Photolysis Measurement.

In the cohort of the present example, the optimal cut point for abnormal photolytic measurement was 1.0 ppb based on Liu's index. FIG. 9 demonstrates the Receiver operator characteristic (ROC) curve. The area under the ROC curve was 0.757. A photolytic measurement greater than or equal to 1.0 ppb had a 70% sensitivity and 76% specificity for identifying a septic patient versus a control (see Table 3, below).

TABLE 3

| Statistical Measures | Photolytic Measurement ≥1 ppb |
|---|---|
| Sensitivity | 0.70 |
| Specificity | 0.76 |
| Area under ROC curve | 0.73 |

The cohort was then dichotomized into normal and abnormal photolytic measurement based on a level of greater than or equal to 1.0 ppb. Patients with a photolytic reading greater than or equal to 1.0 ppb had higher PELOD scores [0.5 (0, 12) versus 12 (10, 21), p-value 0.008], oxygenation indices [0 (0,0) versus 2 (0, 10), p-value 0.011], vasoactive inotrope scores [0 (0, 6) versus 8 (2, 15), p-value 0.002], and higher odds of MODS development [7 (36.8%) versus 15 (79.0%), p-value 0.020] in addition to higher amounts of dysfunctional organs [0 (0, 2) versus 2 (0,3), p-value 0.008]. Patients with photolytic measurements greater than or equal to 1.0 ppb also had fewer ventilator free days, vasoactive free days, and days free from the hospital (see Table 4, below).

TABLE 4
28-day outcomes and severity of illness data
stratified by photolytic measurements.

| | Normal | | Abnormal | | |
|---|---|---|---|---|---|
| | Photolytic | | Photolytic | | |
| | Reading | | Reading | | p- |
| Variable | (<1 ppb) | | (≥1 ppb) | | value |
| PELOD | 0.5 | (0, 12) | 12 | (10, 21) | 0.008 |
| Vasoactive Inotrope Score | 0 | (0, 6) | 8 | (2, 15) | 0.002 |
| Oxygenation Index | 0 | (0, 0) | 2 | (0, 10) | 0.011 |
| ARDS Development | 4 | (21.1%) | 8 | (42.1%) | 0.163 |
| MODS Development | 7 | (36.8%) | 15 | (79.0%) | 0.020 |
| Number of Dysfunctional | 0 | (0, 2) | 2 | (0, 3) | 0.008 |
| Organs | | | | | |

| | Beta- | 95% confidence | |
|---|---|---|---|
| 28-day Outcomes | Coefficient | Interval | p-value |
| Ventilator free days | −5.0 | −9.8 to −0.2 | 0.043 |
| Vasoactive free days | −4.6 | −8.6 to −0.5 | 0.027 |
| ICU free days | −2.9 | −10.0 to 4.2 | 0.408 |
| Hospital free days | −7.0 | −13.4 to −0.5 | 0.035 |

Liu's method was used to determine optimal cut point for normal versus abnormal values. PELOD = Pediatric Logistic Organ Dysfunction score; ARDS = Acute respiratory distress syndrome; MODS = Multiple organ dysfunction syndrome; ICU = Intensive care unit. 28-day outcomes are composite outcomes of days alive and free from ventilator, hospital, ICU, or vasoactive support.

Association of S-Nitrosothiol Levels with Length of ICU Stay.

S-nitrosothiol levels measured by the device were associated with longer lengths of stay data. Specifically, hospital and ICU LOS, as well as length of invasive mechanical ventilation, and length of vasoactive requirement were all increased as S-nitrosothiol levels increased (see Table 5, below).

TABLE 5
Linear regressions of photolytic readings and length of stay

| | Beta- | 95% Confidence | |
|---|---|---|---|
| Variable | coefficient | Interval | p-value |
| Hospital LOS | 271.8 | 25.1 to 518.4 | 0.032 |
| ICU LOS | 331.1 | 13.5 to 648.6 | 0.042 |
| Length of IMV | 126.1 | −165.1 to 417.4 | 0.374 |
| Length of vasoactive | 30.4 | −50.9 to 111.8 | 0.445 |
| support | | | |

LOS = length of stay; IMV = Invasive mechanical ventilation.

Multivariate linear regression models were constructed for S-nitrosothiol levels and length of stay data controlling for whether participants were controls or septic patients. After controlling for the participant cohort type, higher S-nitrosothiol levels trended towards an association with longer length of hospital and ICU stays [Hospital LOS Beta coefficient: 1.4 (−0.0 to 2.7), p-value 0.052; ICU LOS Beta coefficient: 1.2 (−0.2 to 2.5), p-value 0.083].

Linear regression models of photolytic readings also showed statistically significant relationship with certain severity variables, shown in Table 6, below.

TABLE 6
Linear regressions of photolytic readings
and differing severity of illness variables

| | Beta- | 95% confidence | |
|---|---|---|---|
| Variable | Coefficient | interval | p-value |
| Oxygenation Index | 1.6 | 0.5 to 2.6 | 0.004 |
| Vasoactive Inotrope Score | 1.3 | 0.1 to 2.5 | 0.029 |
| PELOD | 1.6 | 0.4 to 2.9 | 0.010 |
| Number of dysfunctional | 1.8 | 0.4 to 3.3 | 0.012 |
| organs | | | |

PELOD = Pediatric Logistic Organ Dysfunction score. Oxygenation index, vasoactive inotrope score, and number of dysfunctional organs was the maximum value noted in the 72 hours after photolytic measurement. Oxygenation index, vasoactive inotrope score and PELOD scores were all log-transformed for analysis. Number of dysfunctional organs was based on Goldstein Criteria.

Association of S-Nitrosothiol Levels with MODS and ARDS Development.

Higher S-nitrosothiol levels were associated with higher odds of both multiple organ dysfunction syndrome (MODS) and acute respiratory distress syndrome (ARDS) development at multiple time points (see Table 7, below). One ppb increase in NO measured by the system of the present disclosure was associated with a 12-fold increased odds of development of MODS in the following 24 hours and 12.9-fold increased likelihood of development of MODS in the following 72 hours (p-values 0.039 and 0.043 respectively). Likewise, a one ppb increase in NO measured by the device was associated with a 15.3-fold increased odds of development of ARDS in the following 24 hours (p-value 0.030).

TABLE 7
Logistic Regressions of photolytic readings and both
development of MODS and development of ARDS within
24 and 72 hours of photolytic measurements.

| | Odds | 95% Confidence | |
|---|---|---|---|
| Variable | Ratio | Interval | p-value |
| MODS 24 | 13.6 | 1.3 to 144.6 | 0.030 |
| MODS 72 | 15.4 | 1.3 to 186.1 | 0.032 |
| ARDS 24 | 12.7 | 1.0 to 155.9 | 0.047 |
| ARDS 72 | 5.7 | 0.7 to 47.7 | 0.110 |

MODS = multiple organ dysfunction syndrome; ARDS = acute respiratory distress syndrome.

### DISCUSSION

This example provides the first study to test the system and method of the present disclosure in a cohort of patients with sepsis. S-nitrosothiol levels as measured by the system were significantly higher in patients with sepsis compared to controls. This study also identified a cut-off value to determine abnormal S-nitrosothiol levels as measured by the system and method. Additionally, when septic patients were dichotomized into normal and abnormal S-nitrosothiol levels, an abnormal S-nitrosothiol level was associated with worse exploratory clinical outcomes.

Similar to what has been demonstrated ex vivo in both animal models and humans, the in vivo models of the present example confirmed that S-nitrosothiol levels were higher in patients with sepsis compared to controls. S-nitrosothiols are produced to counter multiple infections and it has been demonstrated that S-nitrosothiols likely have important roles in both the amelioration and the pathophysiology of sepsis. Doctor et al. previously showed that there are higher levels of S-nitrosylated hemoglobin present in patients with systemic inflammatory response syndrome and acute respiratory distress syndrome. Doctor, A.; Platt, R.; Sheram, M. L.; Eischeid, A.; McMahon, T.; Maxey, T.; Doherty, J.; Axelrod, M.; Kline, J.; Gurka, M.; et al. Hemoglobin conformation couples erythrocyte S-nitrosothiol content to O₂ gradients. Proc Natl Acad Sci USA 2005, 102 (16), 5709-5714. DOI: 10.1073/pnas.0407490102. Liu et al. concurred in this line of investigation, demonstrating higher levels of S-nitrosylated hemoglobin in adult patients with gram-negative sepsis. Liu, L.; Yan, Y.; Zeng, M.; Zhang, J.; Hanes, M. A.; Ahearn, G.; McMahon, T. J.; Dickfeld, T.; Marshall, H. E.; Que, L. G.; et al. Essential roles of S-nitrosothiols in vascular homeostasis and endotoxic shock. Cell 2004, 116 (4), 617-628. DOI: 10.1016/s0092-8674(04)00131-x. The present example adds to these findings by demonstrating higher total S-nitrosothiol levels as measured in the PICU in situ in pediatric patients with sepsis.

Given that S-nitrosothiols play a complex role in sepsis, it is important to attempt to establish normal and abnormal levels of S-nitrosothiols, as shown in the present example. Using Liu's index, an optimal cut-point was determined to stratify patients as having sepsis or not. For the system and method of the present disclosure, the cut-off for the S-nitrosothiol reporter molecule, NO, was found to be 1.0 ppb NO.

In this cohort of patients after dichotomizing the cohort into normal and abnormal S-nitrosothiol levels, a number of clinically relevant outcomes were demonstrated that were worse in the cohort of patients with abnormal S-nitrosothiol levels. Higher oxygenation indices were demonstrated in patients with abnormal S-nitrosothiol levels, which is consistent with prior human studies examining nitrosylated hemoglobin levels in patients with acute respiratory distress syndrome. Similarly, the present example demonstrated worse hemodynamics evidenced by a higher vasoactive inotrope score in those with abnormal S-nitrosothiol levels as measured by the system and method of the present disclosure. This result is also consistent with prior studies. Basal S-nitrosothiol levels have been demonstrated to increase vasodilation in bioassays, and lower blood pressure has been seen when nitrosylated hemoglobin is infused intravenously.

### Conclusion

Using the system and method of the present disclosure, S-nitrosothiol levels were measured in-vivo for a cohort of patients with and without sepsis. Higher S-nitrosothiol levels were seen in patients with sepsis compared to controls. When dichotomized, abnormal S-nitrosothiol levels are associated with worse clinical outcomes.

### Example 3. Measurement of Capillary Bed Perfusion in Disease States Involving Vascular Disease

A subject is identified as having a disease state involving vascular disease, e.g., arterial occlusion, venous occlusion, excessive vasodilation, impaired cardiac function, impaired oxygen delivery, decreased blood oxygen content, or any combination thereof. S-nitrosothiol levels, e.g., in the form of S-nitrosohaemoglobin, of a tissue in the subject are measured using the system of the present disclosure. Erythrocyte haemoglobin is S-nitrosylated in the lung; thus levels of S-nitrosothiols in capillary beds are used as a proxy for perfusion levels. See Jia, L.; Bonaventura, C.; Bonaventura, J.; Stamler, J. S. S-nitrosohaemoglobin: a dynamic activity of blood involved in vascular control. Nature 1996, 380 (6571), 221-226. DOI: 10.1038/380221a0. Higher levels of S-nitrosothiols are associated with high capillary bed perfusion; lower levels of S-nitrosothiols are associated with lower capillary bed perfusion. The S-nitrosothiol measurement is taken one or additional more times if it is determined that such measurement is necessary or helpful for monitoring or treatment.

### Example 4. Monitoring Tissue Oxygenation in Exercise and/or at High Altitude

A subject is identified as needing measurement of blood oxygenation, e.g., during exercise exertion activity, at high altitude, or both. A suitable subject may be engaged in any activity where low blood oxygenation is a known risk, e.g., in an hypoxic environment, scuba diving, etc. S-nitrosothiol levels, e.g., in the form of S-nitrosohaemoglobin, of a tissue in the subject are measured using the system of the present disclosure. Erythrocyte haemoglobin is S-nitrosylated in the lung; thus levels of S-nitrosothiols in capillary beds are used as a proxy for oxygenation levels. Further, oxygenation accelerates S-nitrosylation of haemoglobin. Rates of S-nitrosylation are faster in the oxy conformation (Hb[Fe_{II}]O₂) than in the deoxy state (Hb[Fe_{II}]). See Jia, L.; Bonaventura, C.; Bonaventura, J.; Stamler, J. S. S-nitrosohaemoglobin: a dynamic activity of blood involved in vascular control. Nature 1996, 380 (6571), 221-226. DOI: 10.1038/380221a0; Janocha, A. J.; Koch, C. D.; Tiso, M.; Ponchia, A.; Doctor, A.; Gibbons, L.; Gaston, B.; Beall, C. M.; Erzurum, S. C. Nitric oxide during altitude acclimatization. N Engl J Med 2011, 365, 1942-1944, doi:10.1056/NEJMc1107887. The S-nitrosothiol measurement is taken one or additional more times if it is determined that such measurement is necessary or helpful for monitoring or treatment.

### Example 5. Measuring S-Nitrosothiols Levels to Monitor High-Altitude Acclimatization

A subject is identified as needing measurement of high-altitude acclimatization. Non-limiting examples include mountaineers and pilots. Baseline S-nitrosothiol levels, e.g., in the form of S-nitrosohaemoglobin levels, are measured in the subject using the system of the present disclosure. One or more additional S-nitrosothiol measurements are taken to monitor physiological response to hypoxia, e.g., at high altitude. See Janocha, A. J.; Koch, C. D.; Tiso, M.; Ponchia, A.; Doctor, A.; Gibbons, L.; Gaston, B.; Beall, C. M.; Erzurum, S. C. Nitric oxide during altitude acclimatization. N Engl J Med 2011, 365, 1942-1944, doi:10.1056/NEJMc1107887. Higher S-nitrosothiol levels are associated with acclimatization to the hypoxia. Further S-nitrosothiol measurements are taken if it is determined that such measurements are necessary or helpful for monitoring or treatment.

### REFERENCES

Each of the patents, published patent applications, and non-patent publications referenced in the present disclosure are hereby incorporated by reference in their entireties.

### REFERENCES FOR EXAMPLE 1

Decker, M. J., K. P. Conrad, and K. P. Strohl. Noninvasive oximetry in the rat. Biomedical Instrumentation & Technology 23.3 (1989): 222-228.
De Kock, J. P., et al. The effect of varying LED intensity on pulse oximeter accuracy. Journal of medical engineering & technology 15.3 (1991): 111-115.
Gaston B, Singel D, Doctor A, Stamler J S., S-Nitrosothiol signaling in respiratory biology, Am J Respir Crit Care Med. 173: 1186-1193, 2006.
Gow, A., Doctor, A., Mannick, J., & Gaston, B. S-Nitrosothiol measurements in biological systems. Journal of Chromatography B, 851(1-2), 140-151, 2007.
Haldar S M, Stamler J S., S-nitrosylation: integrator of cardiovascular performance and oxygen delivery, J Clin Invest. 123: 101-110, 2013.
Jaffrey S R, Erdjument-Bromage H, Ferris C D, Tempst P, Snyder S H., Protein S-nitrosylation: a physiological signal for neuronal nitric oxide, Nat Cell Biol. 3:193, 2001.
Marozkina N V, Gaston B., Nitrogen chemistry and lung physiology, Annu Rev Physiol. 77: 431-452, 2015.
Schwarz A. J. et al., The low-frequency blood oxygenation level-dependent functional connectivity signature of the hippocampal-prefrontal network in the rat brain, Neuroscience. 228: 243-258, 2013.
Seckler J M, Meyer N M, Burton S T, Bates J N, Gaston B, and Lewis S J, Detection of trace concentrations of S-nitrosothiols by means of a capacitive sensor, PLoS One. vol. 12, no. 10, October 2017.
Singel D J, Stamler J S, Blood traffic control, Nature. 430: 297, 2004.
Inoue, K.; Akaike, T.; Miyamoto, Y.; Okamoto, T.; Sawa, T.; Otagiri, M.; Suzuki, S.; Yoshimura, T.; Maeda, H. Nitrosothiol formation catalyzed by ceruloplasmin. Implication for cytoprotective mechanism in vivo. J Biol Chem 1999, 274, 27069-27075.
Marozkina, N.; Gaston, B. An Update on Thiol Signaling: S-Nitrosothiols, Hydrogen Sulfide and a Putative Role for Thionitrous Acid. Antioxidants (Basel) 2020, 9, 225, doi:10.3390/antiox9030225.
Doctor, A.; Platt, R.; Sheram, M. L.; Eischeid, A.; McMahon, T.; Maxey, T.; Doherty, J.; Axelrod, M.; Kline, J.; Gurka, M., et al. Hemoglobin conformation couples erythrocyte S-nitrosothiol content to O₂ gradients. Proc Natl Acad Sci USA 2005, 102, 5709-5714, doi:10.1073/pnas.0407490102.
Straub, A. C.; Lohman, A. W.; Billaud, M.; Johnstone, S. R.; Dwyer, S. T.; Lee, M. Y.; Bortz, P. S.; Best, A. K.; Columbus, L.; Gaston, B., et al. Endothelial cell expression of haemoglobin alpha regulates nitric oxide signalling. Nature 2012, 491, 473-477, doi:10.1038/nature11626.
Gaston, B.; Smith, L.; Bosch, J.; Seckler, J.; Kunze, D.; Kiselar, J.; Marozkina, N.; Hodges, C. A.; Wintrobe, P.; McGee, K., et al. Voltage-gated potassium channel proteins and stereoselective S-nitroso-L-cysteine signaling. JCI Insight 2020, 5, doi:10.1172/jci.insight.134174.
Stamler, J. S.; Jia, L.; Eu, J. P.; McMahon, T. J.; Demchenko, I. T.; Bonaventura, J.; Gernert, K.; Piantadosi, C. A. Blood Flow Regulation by S-Nitrosohemoglobin in the Physiological Oxygen Gradient. Science 1997, 276, 2034-2037, doi:10.1126/science.276.5321.2034.
Seckler, J. M.; Shen, J.; Lewis, T. H. J.; Abdulameer, M. A.; Zaman, K.; Palmer, L. A.; Bates, J. N.; Jenkins, M. W.; Lewis, S. J. NADPH diaphorase detects S-nitrosylated proteins in aldehyde-treated biological tissues. Scientific Reports 2020, 10, 21088, doi:10.1038/s41598-020-78107-6.
Hunt, J.; Byrns, R. E.; Ignarro, L. J.; Gaston, B. Condensed expirate nitrite as a home marker for acute asthma. Lancet 1995, 346, 1235-1236.
Gaston, B.; Kelly, R.; Urban, P.; Liu, L.; Henderson, E. M.; Doctor, A.; Teague, W. G.; Fitzpatrick, A.; Erzurum, S.; Hunt, J. F. Buffering airway acid decreases exhaled nitric oxide in asthma. J Allergy Clin Immunol 2006, 118, 817-822, doi:10.1016/j.jaci.2006.06.040.
Janocha, A. J.; Koch, C. D.; Tiso, M.; Ponchia, A.; Doctor, A.; Gibbons, L.; Gaston, B.; Beall, C. M.; Erzurum, S. C. Nitric oxide during altitude acclimatization. N Engl J Med 2011, 365, 1942-1944, doi:10.1056/NEJMc1107887.
Ware, L. B.; Magarik, J. A.; Wickersham, N.; Cunningham, G.; Rice, T. W.; Christman, B. W.; Wheeler, A. P.; Bernard, G. R.; Summar, M. L. Low plasma citrulline levels are associated with acute respiratory distress syndrome in patients with severe sepsis. Crit Care 17, R10.
Gaston, B.; Drazen, J. M.; Jansen, A.; Sugarbaker, D. A.; Loscalzo, J.; Richards, W.; Stamler, J. S. Relaxation of human bronchial smooth muscle by S-nitrosothiols in vitro. J Pharmacol Exp Ther 1994, 268, 978-984.
Stsiapura, V. I.; Shuali, V. K.; Gaston, B. M.; Lehmann, K. K. Detection of S-nitroso compounds by use of midinfrared cavity ring-down spectroscopy. Anal Chem 2015, 87, 3345-3353, doi:10.1021/ac5045143.
Jaffrey, S. R. Detection and characterization of protein nitrosothiols. Methods Enzymol 2005, 396, 105-118, doi:10.1016/S0076-6879(05)96011-4.
Rogers, S. C.; Gibbons, L. B.; Griffin, S.; Doctor, A. Analysis of S-nitrosothiols via copper cysteine (2C) and copper cysteine-carbon monoxide (3C) methods. Methods 2013, 62, 123-129, doi:10.1016/j.ymeth.2012.10.008.
Möller, M. N.; Rios, N.; Trujillo, M.; Radi, R.; Denicola, A.; Alvarez, B. Detection and quantification of nitric oxide-derived oxidants in biological systems. The Journal of biological chemistry 2019, 294, 14776-14802, doi:10.1074/jbc.REV119.006136.
Dweik, R. A.; Laskowski, D.; Abu-Soud, H. M.; Kaneko, F.; Hutte, R.; Stuehr, D. J.; Erzurum, S. C. Nitric oxide synthesis in the lung. Regulation by oxygen through a kinetic mechanism. J Clin Invest 1998, 101, 660-666.
Shin, H. W.; Shelley, D. A.; Henderson, E. M.; Fitzpatrick, A.; Gaston, B.; George, S. C. Airway nitric oxide release is reduced after PBS inhalation in asthma. J Appl Physiol 2007, 102, 1028-1033, doi:10.1152/japplphysiol.01012.2006.
Stsiapura, V. I.; Bederman, I.; Stepuro, I I; Morozkina, T. S.; Lewis, S. J.; Smith, L.; Gaston, B.; Marozkina, N. S-Nitrosoglutathione formation at gastric pH is augmented by ascorbic acid and by the antioxidant vitamin complex, Resiston. Pharm Biol 2018, 56, 86-93, doi:10.1080/13880209.2017.1421674.
How much sun is too much?, InformedHealth.org [Internet]. Cologne, Germany: Institute for Quality and Efficiency in Health Care (IQWiG) 2011, https://www.ncbi.nlm.nih.gov/books/NBK321117/(last update Nov. 29, 2018).
Godar, D. E. U V doses worldwide. Photochem Photobiol 2005, 81, 736-749, doi:10.1562/2004-09-07-ir-308r.1.
Asano, K.; Chee, C. B.; Gaston, B.; Lilly, C. M.; Gerard, C.; Drazen, J. M.; Stamler, J. S. Constitutive and inducible nitric oxide synthase gene expression, regulation, and activity in human lung epithelial cells. Proc Natl Acad Sci USA 1994, 91, 10089-10093.
Gaston, B.; Reilly, J.; Drazen, J. M.; Fackler, J.; Ramdev, P.; Arnelle, D.; Mullins, M. E.; Sugarbaker, D. J.; Chee, C.; Singel, D. J., et al. Endogenous nitrogen oxides and bronchodilator S-nitrosothiols in human airways. Proc Natl Acad Sci USA 1993, 90, 10957-10961.
Hare, J. M. Spatial confinement of isoforms of cardiac nitric-oxide synthase: unravelling the complexities of nitric oxide's cardiobiology. Lancet 2004, 363, 1338-1339, doi:10.1016/S0140-6736(04)16083-2.
Stamler, J. S.; Singel, D. J.; Loscalzo, J. Biochemistry of nitric oxide and its redox-activated forms. Science 1992, 258, 1898-1902.
Forrester, M. T.; Seth, D.; Hausladen, A.; Eyler, C. E.; Foster, M. W.; Matsumoto, A.; Benhar, M.; Marshall, H. E.; Stamler, J. S. Thioredoxin-interacting protein (Txnip) is a feedback regulator of S-nitrosylation. J Biol Chem 2009, 284, 36160-36166, doi:10.1074/jbc.M109.057729.
Simon, D. I.; Mullins, M. E.; Jia, L.; Gaston, B.; Singel, D. J.; Stamler, J. S. Polynitrosylated proteins: characterization, bioactivity, and functional consequences. Proc Natl Acad Sci USA 1996, 93, 4736-4741.
Fang, K.; Ragsdale, N. V.; Carey, R. M.; MacDonald, T.; Gaston, B. Reductive assays for S-nitrosothiols: implications for measurements in biological systems. Biochem Biophys Res Commun 1998, 252, 535-540, doi:10.1006/bbrc.1998.9688.

### REFERENCES FOR EXAMPLE 2

Rudd, K. E.; Johnson, S. C.; Agesa, K. M.; Shackelford, K. A.; Tsoi, D.; Kievlan, D. R.; Colombara, D. V.; Ikuta, K. S.; Kissoon, N.; Finfer, S.; et al. Global, regional, and national sepsis incidence and mortality, 1990-2017: analysis for the Global Burden of Disease Study. Lancet 2020, 395 (10219), 200-211. DOI: 10.1016/S0140-6736(19)32989-7 From NLM Medline.
Dugani, S.; Veillard, J.; Kissoon, N. Reducing the global burden of sepsis. CMAJ 2017, 189 (1), E2-E3. DOI: 10.1503/cmaj.160798 From NLM Medline.
Fleischmann-Struzek, C.; Goldfarb, D. M.; Schlattmann, P.; Schlapbach, L. J.; Reinhart, K.; Kissoon, N. The global burden of paediatric and neonatal sepsis: a systematic review. Lancet Respir Med 2018, 6 (3), 223-230. DOI: 10.1016/S2213-2600(18)30063-8 From NLM Medline.
Coletta, C.; Modis, K.; Olah, G.; Brunyanszki, A.; Herzig, D. S.; Sherwood, E. R.; Ungvari, Z.; Szabo, C. Endothelial dysfunction is a potential contributor to multiple organ failure and mortality in aged mice subjected to septic shock: preclinical studies in a murine model of cecal ligation and puncture. Crit Care 2014, 18 (5), 511. DOI: 10.1186/s13054-014-0511-3 From NLM Medline.
Lee, W. L.; Liles, W. C. Endothelial activation, dysfunction and permeability during severe infections. Curr Opin Hematol 2011, 18 (3), 191-196. DOI: 10.1097/MOH.0b013e328345a3d1 From NLM Medline.
Neri, M.; Riezzo, I.; Pomara, C.; Schiavone, S.; Turillazzi, E. Oxidative-Nitrosative Stress and Myocardial Dysfunctions in Sepsis: Evidence from the Literature and Postmortem Observations. Mediators Inflamm 2016, 2016, 3423450. DOI: 10.1155/2016/3423450 From NLM Medline.
Kumar, S.; Gupta, E.; Srivastava, V. K.; Kaushik, S.; Saxena, J.; Goyal, L. K.; Mehta, S.; Jyoti, A. Nitrosative stress and cytokines are linked with the severity of sepsis and organ dysfunction. Br J Biomed Sci 2019, 76 (1), 29-34. DOI: 10.1080/09674845.2018.1543160 From NLM Medline.
Chelkeba, L.; Ahmadi, A.; Abdollahi, M.; Najafi, A.; Ghadimi, M. H.; Mosaed, R.; Mojtahedzadeh, M. The effect of parenteral selenium on outcomes of mechanically ventilated patients following sepsis: a prospective randomized clinical trial. Ann Intensive Care 2015, 5 (1), 29. DOI: 10.1186/s13613-015-0071-y From NLM PubMed-not-MEDLINE.
Lopez, A.; Lorente, J. A.; Steingrub, J.; Bakker, J.; McLuckie, A.; Willatts, S.; Brockway, M.; Anzueto, A.; Holzapfel, L.; Breen, D.; et al. Multiple-center, randomized, placebo-controlled, double-blind study of the nitric oxide synthase inhibitor 546C88: effect on survival in patients with septic shock. Crit Care Med 2004, 32 (1), 21-30. DOI: 10.1097/01.CCM.0000105581.01815.C6 From NLM Medline.
Pascual-Ramirez, J.; Koutrouvelis, A. The nitric oxide pathway antagonists in septic shock: Meta-analysis of controlled clinical trials. J Crit Care 2019, 51, 34-38. DOI: 10.1016/j.jcrc.2019.01.013 From NLM PubMed-not-MEDLINE.
Gaston, B.; Singel, D.; Doctor, A.; Stamler, J. S. S-nitrosothiol signaling in respiratory biology. Am J Respir Crit Care Med 2006, 173 (11), 1186-1193. DOI: 10.1164/rccm.200510-1584PP From NLM Medline.
Haldar, S. M.; Stamler, J. S. S-nitrosylation: integrator of cardiovascular performance and oxygen delivery. J Clin Invest 2013, 123 (1), 101-110. DOI: 10.1172/JCI62854 From NLM Medline.
Marozkina, N. V.; Gaston, B. Nitrogen chemistry and lung physiology. Annu Rev Physiol 2015, 77, 431-452. DOI: 10.1146/annurev-physiol-021113-170352 From NLM Medline.
Singel, D. J.; Stamler, J. S. Blood traffic control. Nature 2004, 430 (6997), 297. DOI: 10.1038/430297a From NLM Medline.
Stamler, J. S.; Jia, L.; Eu, J. P.; McMahon, T. J.; Demchenko, I. T.; Bonaventura, J.; Gernert, K.; Piantadosi, C. A. Blood flow regulation by S-nitrosohemoglobin in the physiological oxygen gradient. Science 1997, 276 (5321), 2034-2037. DOI: 10.1126/science.276.5321.2034 From NLM Medline.
Inoue, K.; Akaike, T.; Miyamoto, Y.; Okamoto, T.; Sawa, T.; Otagiri, M.; Suzuki, S.; Yoshimura, T.; Maeda, H. Nitrosothiol formation catalyzed by ceruloplasmin. Implication for cytoprotective mechanism in vivo. J Biol Chem 1999, 274 (38), 27069-27075. DOI: 10.1074/jbc.274.38.27069 From NLM Medline.
Doctor, A.; Platt, R.; Sheram, M. L.; Eischeid, A.; McMahon, T.; Maxey, T.; Doherty, J.; Axelrod, M.; Kline, J.; Gurka, M.; et al. Hemoglobin conformation couples erythrocyte S-nitrosothiol content to O₂ gradients. Proc Natl Acad Sci USA 2005, 102 (16), 5709-5714. DOI: 10.1073/pnas.0407490102 From NLM Medline.
Gaston, B.; Smith, L.; Bosch, J.; Seckler, J.; Kunze, D.; Kiselar, J.; Marozkina, N.; Hodges, C. A.; Wintrobe, P.; McGee, K.; et al. Voltage-gated potassium channel proteins and stereoselective S-nitroso-L-cysteine signaling. JCI Insight 2020, 5 (18). DOI: 10.1172/jci.insight.134174 From NLM Medline.
Gaston, B.; Reilly, J.; Drazen, J. M.; Fackler, J.; Ramdev, P.; Arnelle, D.; Mullins, M. E.; Sugarbaker, D. J.; Chee, C.; Singel, D. J.; et al. Endogenous nitrogen oxides and bronchodilator S-nitrosothiols in human airways. Proc Natd Acad Sci USA 1993, 90 (23), 10957-10961. DOI: 10.1073/pnas.90.23.10957 From NLM Medline.
Neidigh, N.; Alexander, A.; van Emmerik, P.; Higgs, A.; Plack, L.; Clem, C.; Cater, D.; Marozkina, N.; Gaston, B. Photolytic Measurement of Tissue S-Nitrosothiols in Rats and Humans In Vivo. Molecules 2022, 27 (4). DOI: 10.3390/molecules27041294 From NLM Medline.
Gaies, M. G.; Gurney, J. G.; Yen, A. H.; Napoli, M. L.; Gajarski, R. J.; Ohye, R. G.; Charpie, J. R.; Hirsch, J. C. Vasoactive-inotropic score as a predictor of morbidity and mortality in infants after cardiopulmonary bypass. Pediatr Crit Care Med 2010, 11 (2), 234-238. DOI: 10.1097/PCC.0b013e3181b806fc.
Wernovsky, G.; Wypij, D.; Jonas, R. A.; Mayer, J. E., Jr.; Hanley, F. L.; Hickey, P. R.; Walsh, A. Z.; Chang, A. C.; Castaneda, A. R.; Newburger, J. W.; et al. Postoperative course and hemodynamic profile after the arterial switch operation in neonates and infants. A comparison of low-flow cardiopulmonary bypass and circulatory arrest. Circulation 1995, 92 (8), 2226-2235. DOI: 10.1161/01.cir.92.8.2226.
Emeriaud, G.; Lopez-Fernandez, Y. M.; Iyer, N. P.; Bembea, M. M.; Agulnik, A.; Barbaro, R. P.; Baudin, F.; Bhalla, A.; Brunow de Carvalho, W.; Carroll, C. L.; et al. Executive Summary of the Second International Guidelines for the Diagnosis and Management of Pediatric Acute Respiratory Distress Syndrome (PALICC-2). Pediatr Crit Care Med 2023, 24 (2), 143-168. DOI: 10.1097/PCC.0000000000003147.
Goldstein, B.; Giroir, B.; Randolph, A.; International Consensus Conference on Pediatric, S. International pediatric sepsis consensus conference: definitions for sepsis and organ dysfunction in pediatrics. Pediatr Crit Care Med 2005, 6 (1), 2-8. DOI: 10.1097/01.PCC.0000149131.72248.E6.
Schisterman, E. F.; Perkins, N. J.; Liu, A.; Bondell, H. Optimal cut-point and its corresponding Youden Index to discriminate individuals using pooled blood samples. Epidemiology 2005, 16 (1), 73-81. DOI: 10.1097/01.ede.0000147512.81966.ba.
(26) StataCorp. 2021. Stata Statistical Software: Release 17. College Station, T X: StataCorp LLC.
Jourd'heuil, D.; Gray, L.; Grisham, M. B. S-nitrosothiol formation in blood of lipopolysaccharide-treated rats. Biochem Biophys Res Commun 2000, 273 (1), 22-26. DOI: 10.1006/bbrc.2000.2892 From NLM Medline.
Liu, L.; Yan, Y.; Zeng, M.; Zhang, J.; Hanes, M. A.; Ahearn, G.; McMahon, T. J.; Dickfeld, T.; Marshall, H. E.; Que, L. G.; et al. Essential roles of S-nitrosothiols in vascular homeostasis and endotoxic shock. Cell 2004, 116 (4), 617-628. DOI: 10.1016/s0092-8674(04)00131-x From NLM Medline.
De Groote, M. A.; Testerman, T.; Xu, Y.; Stauffer, G.; Fang, F. C. Homocysteine antagonism of nitric oxide-related cytostasis in Salmonella typhimurium. Science 1996, 272 (5260), 414-417. DOI: 10.1126/science.272.5260.414 From NLM Medline.
de Jesus-Berrios, M.; Liu, L.; Nussbaum, J. C.; Cox, G. M.; Stamler, J. S.; Heitman, J. Enzymes that counteract nitrosative stress promote fungal virulence. Curr Biol 2003, 13 (22), 1963-1968. DOI: 10.1016/j.cub.2003.10.029 From NLM Medline.
MacMicking, J. D.; North, R. J.; LaCourse, R.; Mudgett, J. S.; Shah, S. K.; Nathan, C. F. Identification of nitric oxide synthase as a protective locus against tuberculosis. Proc Natl Acad Sci USA 1997, 94 (10), 5243-5248. DOI: 10.1073/pnas.94.10.5243 From NLM Medline.
McMahon, T. J.; Moon, R. E.; Luschinger, B. P.; Carraway, M. S.; Stone, A. E.; Stolp, B. W.; Gow, A. J.; Pawloski, J. R.; Watke, P.; Singel, D. J.; et al. Nitric oxide in the human respiratory cycle. Nat Med 2002, 8 (7), 711-717. DOI: 10.1038/nm718 From NLM Medline.
Pawloski, J. R.; Hess, D. T.; Stamler, J. S. Export by red blood cells of nitric oxide bioactivity. Nature 2001, 409 (6820), 622-626. DOI: 10.1038/35054560 From NLM Medline.
Jia, L.; Bonaventura, C.; Bonaventura, J.; Stamler, J. S. S-nitrosohaemoglobin: a dynamic activity of blood involved in vascular control. Nature 1996, 380 (6571), 221-226. DOI: 10.1038/380221a0 From NLM Medline.

What is claimed is:

1. A system for determining levels of an S-nitrosothiol in situ by measuring nitric oxide released from a target site in the body of a mammal after exposure to ultraviolet light, comprising:
a housing having a proximal end and a distal end and defining a cavity therebetween, the housing further defining an opening at the distal end, wherein the housing is configured for placement of the distal end in proximity with the target site to form a seal between the distal end of the housing and the target site, wherein a portion of the target site is exposed to the cavity through the opening;
a source of ultraviolet light affixed to the proximal end of the housing and positioned to irradiate the portion of the target site through the opening; and
a nitric oxide analyzer configured to receive a gas sample from the cavity following irradiation of the target site and operable to measure a nitric oxide concentration in the gas sample.

2. The system of claim 1, wherein the levels of the S-nitrosothiol in situ are determined based on a correlation with the nitric oxide concentration.

3. The system of claim 1 wherein the target site is one or more of an organ, tissue, body cavity, and body fluid.

4. The system of claim 1, wherein the target site is one or more of skin, oral cavity, and spinal fluid.

5. The system of claim 1 further comprising a lens movably affixed to the housing, the lens operable to focus the source of ultraviolet light upon the target site.

6. The system of claim 5, wherein the lens is a double convex focusing lens having an effective focal length of from about 12 mm to about 36 mm and a diameter of from about 6 mm to about 18 mm.

7. The system of claim 1, wherein the ultraviolet light comprises light having a wavelength of from about 300 to about 375 nm.

8. The system of claim 1, wherein the nitric oxide analyzer performs a chemiluminescence-based assay.

9. The system of claim 1, wherein the S-nitrosothiol is S-nitroso-L-cysteine or S-nitrosoglutathione.

10. The system of claim 3, wherein the organ or tissue is an organ or tissue having high vascularization.

11. The system of claim 4, wherein the skin is the skin of a finger or an ear.

12. The system of claim 1, which is operable to diagnose or monitor a disease or condition.

13. The system of claim 12, wherein the disease or condition is sepsis.

14. The system of claim 13, wherein the system is further operable to diagnostically exclude that the disease or condition is cardiogenic shock.

15. A method of determining levels of an S-nitrosothiol in situ by measuring nitric oxide released from a target site in the body of a mammal after exposure to ultraviolet light, comprising:
irradiating the target site with ultraviolet light to generate nitric oxide from the S-nitrosothiol;
receiving the nitric oxide; and
measuring the nitric oxide.

16. The method of claim 15, wherein the levels of the S-nitrosothiol in situ are determined based on a correlation with the nitric oxide concentration.

17. The method of claim 15, wherein the irradiating with ultraviolet light involves focusing the ultraviolet light using a lens.

18. The method of claim 17, wherein the lens is a double convex focusing lens effective focal length of from about 12 mm to about 36 mm and a diameter of from about 6 mm to about 18 mm.

19. The method of claim 17, wherein the ultraviolet light comprises light having a wavelength of from about 300 to about 375 nm.

20. The method of claim 15, wherein the target site is one or more of skin, oral cavity, and spinal fluid.

21. The method of claim 15, wherein the irradiating with the ultraviolet light the target site is performed for a duration of from about 1 to about 30 seconds.
The method of claim 15, wherein the measuring the nitric oxide is performed by a nitric oxide analyzer.

22. The method of claim 15, wherein determining the levels of the S-nitrosothiol is performed to diagnose or monitor a disease or condition.

23. The method of claim 22, wherein the disease or condition is sepsis.

24. A method of monitoring the effects of a medical treatment in a patient in need thereof comprising: treating the patient, then performing the method of claim 22.

25. A method of measuring capillary bed perfusion in a subject in need thereof, comprising: using the system of claim 1 to measure S-nitrosothiol levels in the capillary bed of a tissue of the subject.

26. A method of monitoring tissue oxygenation in a subject in need thereof, comprising: using the system of claim 1 to measure the subject's tissue S-nitrosothiol levels.

27. A method of monitoring high-altitude acclimatization in a subject in need thereof, comprising: using the system of claim 1 to measure a baseline level of the subject's tissue S-nitrosohaemoglobin levels circulating in the subject's blood; and performing one or more comparison measurements of the subject's tissue S-nitrosohaemoglobin levels circulating in the subject's blood.

28. A method of distinguishing sepsis from cardiogenic shock in a patient in need thereof, the method comprising:
determining levels of S-nitrosothiol in situ by measuring nitric oxide released from a target site in the patient's body after exposure to ultraviolet light, comprising:
irradiating the target site with ultraviolet light to generate nitric oxide from the S-nitrosothiol,
receiving the nitric oxide, and
measuring the nitric oxide concentration; and
based on the nitric oxide concentration measurement, determining whether the patient has sepsis of cardiogenic shock.
